(19) United States
(12) Patent Application Publication
(10) Pub. No.: US 2025/0288260 A1
(43) Pub. Date: Sep. 18, 2025

(54) X-RAY CT APPARATUS, CONTROLLER OF X-RAY CT APPARATUS, AND CONTROL METHOD FOR X-RAY CT APPARATUS

(71) Applicant: CANON MEDICAL SYSTEMS CORPORATION, Otawara-shi (JP)

(72) Inventors: Kensuke TAKANASHI, Otawara (JP); Takahito WATANABE, Nasushiobara (JP)

(73) Assignee: CANON MEDICAL SYSTEMS CORPORATION, Otawara-shi (JP)

(21) Appl. No.: 19/077,234

(22) Filed: Mar. 12, 2025

(30) Foreign Application Priority Data
Mar. 18, 2024 (JP) 2024-042130
Jul. 11, 2024 (JP) 2024-111808
Feb. 17, 2025 (JP) 2025-023495

(51) Int. Cl. A61B 6/03; A61B 6/00

(52) U.S. Cl. CPC A61B 6/032; A61B 6/542

(57) ABSTRACT

In one embodiment, an X-ray CT apparatus can perform a tube-current modulation scan. The X-ray CT apparatus includes an X-ray tube, a memory, an X-ray tube power supply, processing circuitry, and a filament control circuit. The memory stores, for each tube voltage to be applied to the X-ray tube, characteristic data in which a filament current flowing through a filament in the X-ray tube and the tube current are associated with each other. The X-ray tube power supply applies the tube voltage to the X-ray tube by periodically switching the tube voltage between a first value and a second value lower than the first value. The processing circuitry updates the command value of the tube current at a predetermined timing according to switching of the tube voltage. The filament control circuit specifies a filament-current command-value corresponding to the command value of the tube current based on the characteristic data.

### CROSS-REFERENCE TO RELATED APPLICATION

This application is based upon and claims the benefit of priority from Japanese Patent Application No. 2024-042130, filed on Mar. 18, 2024, No. 2024-111808, filed on Jul. 11, 2024, and No. 2025-023495, filed on Feb. 17, 2025, the entire contents of which are incorporated herein by reference.

### FIELD

Disclosed embodiments relate to an X-ray CT apparatus, a controller of the X-ray CT apparatus, and a control method for the X-ray CT apparatus.

### BACKGROUND

In an X-ray CT (Computed Tomography) apparatus, a spectral scan method is known as a technique in which image data are acquired by alternately switching a tube voltage to be applied to an X-ray tube between a high tube-voltage and a low tube-voltage during rotation of the X-ray tube inside a gantry.

In the spectral scan method, the tube voltage is switched at high speed during scanning. Thus, if feedback control of the tube current (milliampere) is always performed in the spectral scan method, the value obtained by dividing the tube current value by the filament current value becomes unstable. Hereafter, the feedback control of the tube current is referred to as “the tube-current feedback-control”. Hence, the tube current is stabilized by performing the tube-current feedback-control only in each period during which the tube voltage is high (hereinafter referred to as “the high tube-voltage period”). In each period during which the tube voltage is low (hereinafter referred to as “the low tube-voltage period”), the filament current value commanded at the end of the high tube-voltage period is maintained.

The above-described tube-current feedback-control is effective when the tube current is constant. In the case of a scan in which the tube current is modulated, the filament current during the low tube-voltage period follows the filament current value at the end of the high tube-voltage period, and thus, the tube current decreases by the decrement of the lowered tube voltage. If the high tube-voltage period begins in such a state, the tube current becomes unstable.

Because a tube-current command-signal is not synchronized with a spectral signal, depending on the switching timing, during the high tube-voltage period, the tube voltage may switch to the low tube-voltage before the tube current reaches the command value. After that, the tube current becomes unstable, and there is a concern that this instability may affect image quality.

FIG. 9 is a timing chart illustrating temporal changes in each signal and each detection value in the tube-current feedback-control according to the conventional technology. As shown in FIG. 9, the tube-voltage detection-value and the tube-current feedback-control are switched depending on the spectral signal S1.

When the tube-current command-signal S2 is updated during the high tube-voltage period (High kV), the tube-current detection-value does not reach the command value during the high tube-voltage period, and the tube voltage transitions to the low tube-voltage period (Low kV) in the state where the filament-current command-signal S3 is at its maximum, as shown by the period T1 in FIG. 9.

When the tube-current command signal S2 is updated during the low tube-voltage period, though the tube-current feedback-control is turned on during the next high tube-voltage period, the difference between the tube-current command-value and the tube-current detection-value becomes large, and thus, it takes time for the detection value to converge to the command value, as shown by the period T2 in FIG. 9.

Furthermore, as shown in FIG. 10, when the above-described tube-current feedback-control is performed, the tube-current detection-value becomes lower than a tube-current command-value in the low tube-voltage period. This is because, as shown in FIG. 11, due to characteristics of the X-ray tube, if a filament current value IF1 corresponding to a tube-current value I1 in the high tube-voltage period is maintained, a tube-current value I2 corresponding to the same filament current value IF1 becomes lower than the tube-current value I1 in the low tube-voltage period.

As shown in FIG. 12, when the tube-current feedback-control is performed at all times, the tube-current detection-value becomes much higher than the tube-current command-value when the low tube-voltage period transitions to the high tube-voltage period, thus causing unnecessary radiation exposure of a patient or a user. A description will be given in this regard with reference to FIG. 11. According to the characteristics of the X-ray tube, in the case where the tube-current command-value is set to I1, when the tube-current value converges to I1 in the high tube-voltage period, the filament current value IF1 flows. Next, when the tube-current value converges to I1 after the high tube-voltage period transitions to the low tube-voltage period, a filament current value IF2 flows. Then, when the low tube-voltage period transitions to the high tube-voltage period, due to the characteristics of the X-ray tube at the high tube-voltage, a tube-current value I3 flows according to the filament current value IF2 at the time. That is, the tube-current value is higher than the original tube-current command-value I1 by (I3−I1).

### BRIEF DESCRIPTION OF THE DRAWINGS

In the accompanying drawings:

FIG. 1 is a block diagram illustrating a configuration of an X-ray CT apparatus according to a first embodiment and a second embodiment;

FIG. 2 is a block diagram illustrating a configuration of an X-ray generation system according to the first embodiment;

FIG. 3 is a graph illustrating characteristics between a filament current and a tube current according to the first embodiment;

FIG. 4 is a timing chart illustrating temporal changes in each signal and each detection value according to the first embodiment;

FIG. 5 is another timing chart illustrating temporal changes in each signal and each detection value according to the first embodiment;

FIG. 6 is a block diagram illustrating a configuration of an X-ray generation system according to the second embodiment;

FIG. 7 is a graph illustrating characteristics between a filament current and a tube current according to the second embodiment;

FIG. 8 is a timing chart illustrating temporal changes in each signal and each detection value according to the second embodiment;

FIG. 9 is a timing chart illustrating temporal changes in each signal and each detection value according to the conventional technology;

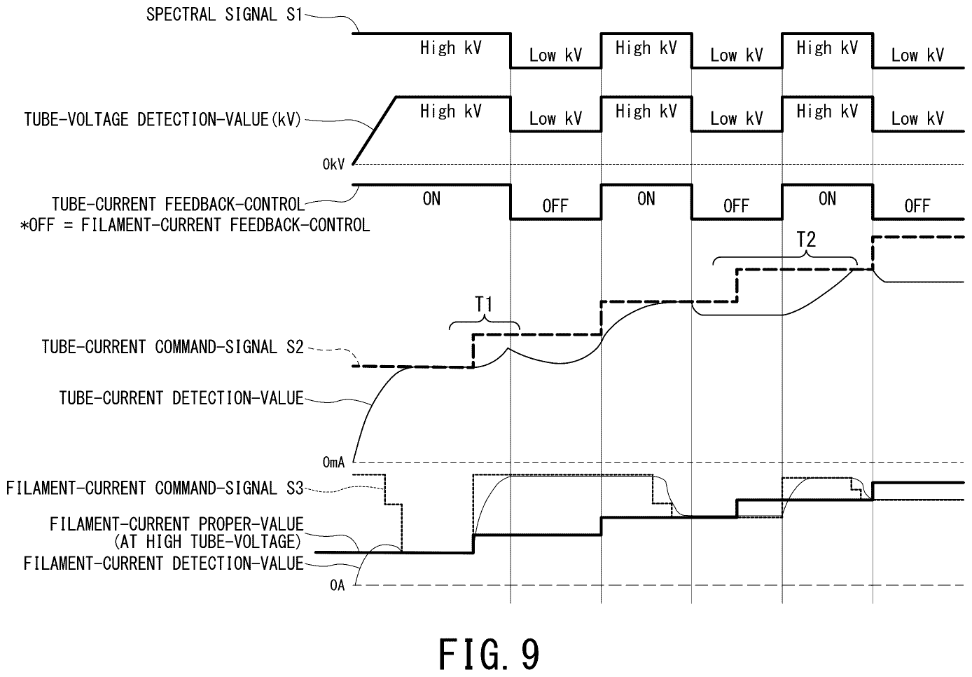

FIG. 10 is a timing chart illustrating temporal changes in each signal and each detection value according to the conventional technology;

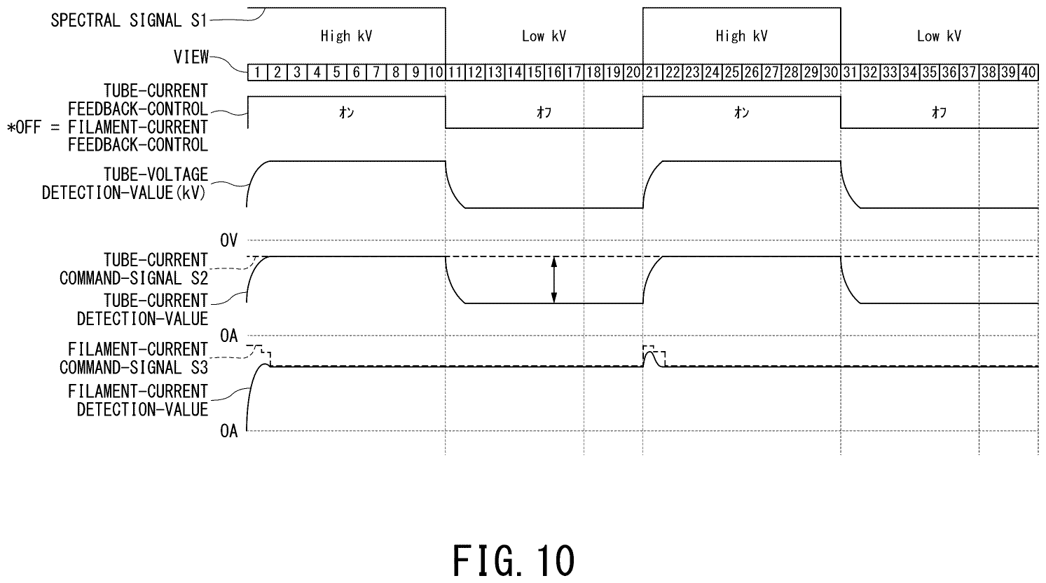

FIG. 11 is a graph illustrating characteristics between a filament current and a tube current according to the conventional technology; and

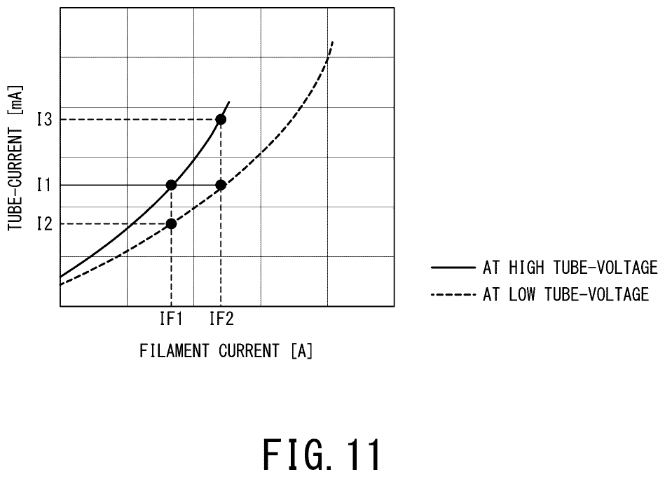

FIG. 12 is a timing chart illustrating temporal changes in each signal and each detection value according to the conventional technology.

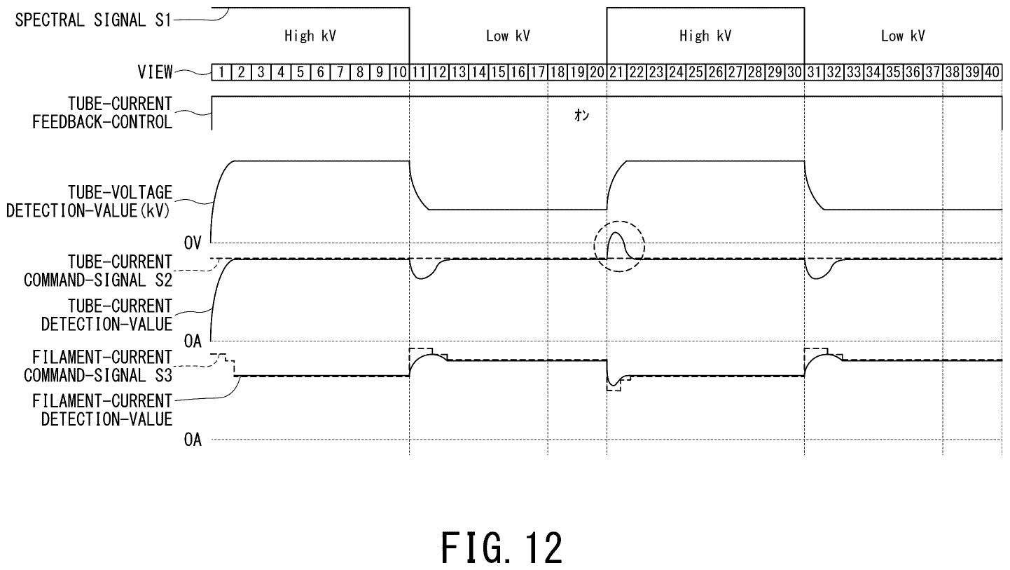

### DETAILED DESCRIPTION

Hereinbelow, embodiments of an X-ray CT apparatus, a controller of the X-ray CT apparatus and a control method for the X-ray CT apparatus will be described in detail by referring to the accompanying drawings.

In one embodiment, an X-ray CT apparatus can perform a tube-current modulation scan. The X-ray CT apparatus includes an X-ray tube, a memory, an X-ray tube power supply, processing circuitry, and a filament control circuit. The X-ray tube irradiates an object with X-rays. The memory stores, for each tube voltage to be applied to the X-ray tube, characteristic data in which a filament current flowing through a filament in the X-ray tube and the tube current are associated with each other. The X-ray tube power supply applies the tube voltage to the X-ray tube by periodically switching the tube voltage between a first tube-voltage value and a second tube-voltage value lower than the first tube-voltage value. The processing circuitry updates the command value of the tube current at a predetermined timing according to switching of the tube voltage. The filament control circuit specifies a filament-current command-value corresponding to the command value of the tube current based on the characteristic data.

### First Embodiment

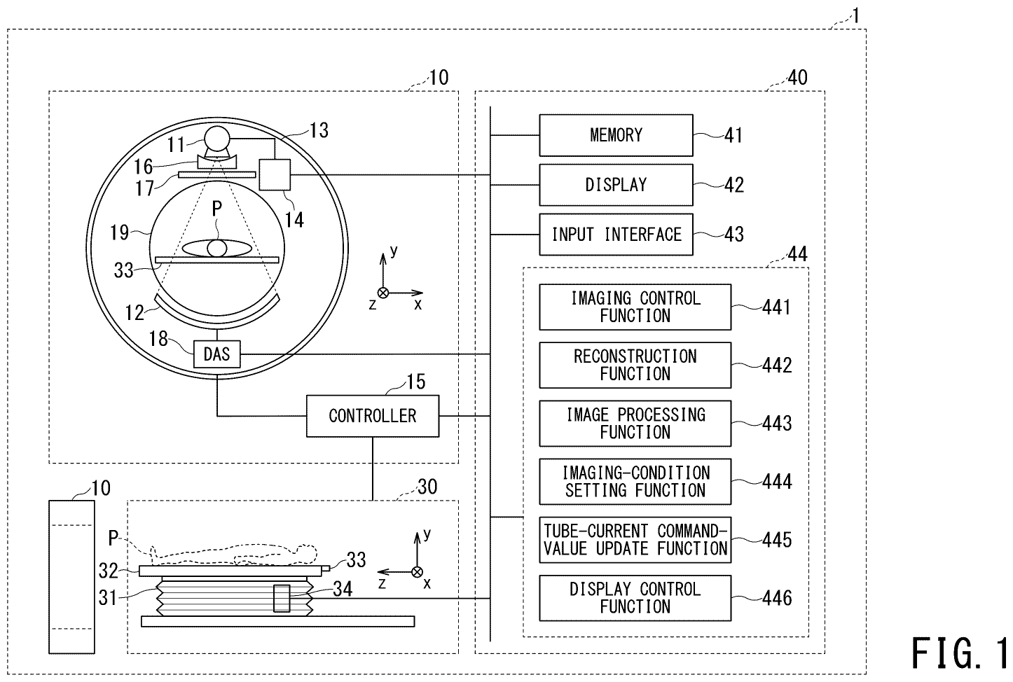

FIG. 1 is a block diagram illustrating a configuration of an X-ray CT apparatus 1 according to a first embodiment. As shown in FIG. 1, the X-ray CT apparatus 1 includes a gantry 10, a bed 30, and a console 40. Although FIG. 1 shows two gantries 10 for facilitating understanding, in reality, there can be only one, three, or more gantries. The X-ray CT apparatus 1 can perform a spectral scan. The spectral scan is one example of a tube-current modulation scan.

The gantry 10 is a scanner having a configuration for X-ray CT imaging of an object P. The bed 30 is a conveying device for placing the object P to be subjected to X-ray CT imaging and for positioning the object P. The console 40 is a computer that controls the gantry 10. For example, the gantry 10 and the bed 30 are installed in a CT examination room, and the console 40 is installed in a control room adjacent to the CT examination room. The gantry 10, the bed 30, and the console 40 are communicably interconnected by wire or wirelessly. Note that the console 40 does not necessarily have to be installed in the control room. For example, all of the console 40, the gantry 10, and the bed 30 may be installed in the same room. The console 40 may also be integrated into the gantry 10.

As shown in FIG. 1, the gantry 10 includes an X-ray tube 11, an X-ray detector 12, a rotating frame 13, an X-ray high-voltage device 14, a controller 15, a wedge 16, a collimator 17, and a data acquisition system (DAS) 18.

The X-ray tube 11 irradiates the object P with X-rays. The X-ray tube 11 includes: a cathode configured to generate thermal electrons; an anode configured to receive the thermal electrons flying from the cathode and generate X-rays; and a vacuum tube configured to hold the cathode and the anode. The X-ray tube 11 is connected to the X-ray high-voltage device 14 via a high-voltage cable. A tube voltage is applied between the cathode and the anode by the X-ray high-voltage device 14. Application of the tube voltage causes the thermal electrons to fly from the cathode to the anode. As the thermal electrons fly, a tube current flows between the cathode and the anode. As the thermal electrons collide with the anode, this collision generates X-rays.

The X-ray detector 12 detects the X-rays having been emitted from the X-ray tube 11 and having passed through the object P, and outputs an electrical signal corresponding to the detected X-ray dose to the DAS 18. The X-ray detector 12 has a structure in which a plurality of X-ray detection element rows are arranged in the slice direction (i.e., in the row direction), and each X-ray detection element row has a plurality of X-ray detection elements arranged in the channel direction.

For example, the X-ray detector 12 is an indirect conversion type detector having a grid, a scintillator array, and a photosensor array. The scintillator array has a plurality of scintillators. Each scintillator outputs the amount of light corresponding to the amount of incident X-rays. The grid is disposed on the side of the X-ray incident surface of the scintillator array, and has an X-ray shielding plate configured to absorb scattered X-rays. The grid is sometimes called a collimator (one-dimensional collimator or two-dimensional collimator). The photosensor array converts the light from each scintillator into an electrical signal that corresponds to the amount of this light. For example, a photodiode is used as the photosensor. The X-ray detector 12 may be a direct conversion type detector.

The rotating frame 13 is an annular frame that supports the X-ray tube 11 and the X-ray detector 12 in such a manner that both can rotate around the rotation axis (i.e., the Z-axis). The rotating frame 13 supports the X-ray tube 11 and the X-ray detector 12 such that both face each other. The rotating frame 13 is supported by a fixed frame (not shown) so as to be rotatable around the rotation axis. The controller 15 rotates the rotating frame 13 around the rotation axis, and thereby, the X-ray tube 11 and the X-ray detector 12 rotate around the rotation axis. The rotating frame 13 rotates at a constant angular velocity around its rotation axis by being supplied with power from a drive mechanism of the controller 15. An image field of view (FOV) is set to an aperture 19 in the rotating frame 13.

In the present embodiment, the rotation axis of the rotating frame 13 in a non-tilted state or the longitudinal direction of a table 33 of the bed 30 is defined as the Z-axis direction, the axial direction orthogonal to the Z-axis direction and horizontal to the floor surface is defined as the X-axis direction, and the axial direction orthogonal to the Z-axis direction and perpendicular to the floor surface is defined as the Y-axis direction.

The X-ray high-voltage device 14 has a high-voltage generator and an X-ray controller. The high-voltage generator has an electric circuit including a transformer and a rectifier, and generates the high voltage to be applied to the X-ray tube 11 and the filament current to be supplied to the X-ray tube 11. The X-ray controller controls the output voltage depending on X-rays emitted by the X-ray tube 11. The high-voltage generator may be of the transformer type or the inverter type. The X-ray high-voltage device 14 may be provided on the rotating frame 13 inside the gantry 10 or may be provided on a fixed frame (not shown) inside the gantry 10.

The wedge 16 adjusts dose of X-rays to be radiated to the object P. The wedge 16 attenuates the X-rays such that the dose of X-rays radiated from the X-ray tube 11 to the object P has a predetermined distribution. For example, aspects of the wedge 16 include a wedge filter, a bow-tie filter, and a metal plate such as aluminum.

The collimator 17 limits the irradiation range of the X-rays that have passed through the wedge 16. The collimator 17 slidably supports a plurality of lead plates that block X-rays, and adjusts the shape of the slits formed by the plurality of lead plates. The collimator 17 is sometimes called an X-ray aperture.

The DAS 18 reads out an electrical signal corresponding to the X-ray dose detected by the X-ray detector 12 from the X-ray detector 12. The DAS 18 amplifies the read-out electrical signal and integrates the electrical signal over a view period so as to acquire detection data having digital values corresponding to the X-ray dose over the view period. The detection data are referred to as projection data. The DAS 18 is realized by an application specific integrated circuit (ASIC) provided with circuit elements that can generate the projection data, for example. The projection data are transmitted to the console 40 via a non-contact data transmission device, for example.

In the present embodiment, the integral type X-ray detector 12 and the X-ray CT apparatus 1 provided with the integral type X-ray detector 12 will be illustrated and described. The techniques according to the present embodiment can also be applied to a photon-counting type X-ray detector.

The controller 15 controls the X-ray high-voltage device 14 and/or the DAS 18 in order to perform X-ray CT imaging on the basis of an imaging control function 441 of processing circuitry 44 of the console 40. The controller 15 includes: processing circuitry including a central processing unit (CPU) or a micro-processing unit (MPU); and a drive mechanism including a motor and an actuator. The processing circuitry has: a processor such as a CPU; and a memory such as a read only memory (ROM) and a random access memory (RAM), as its hardware resources. The controller 15 executes various functions by using a processor configured to execute programs developed in the memory. Note that the various functions are not limited to an aspect of being achieved by single processing circuitry. It may be configured such that the processing circuitry is composed of a plurality of independent processors and each function is implemented by causing each processor to execute the program. The controller 15 may also be achieved by using an ASIC and/or a field programmable gate array (FPGA).

The controller 15 may also be achieved by another complex programmable logic device (CPLD) or a simple programmable logic device (SPLD). The controller 15 has a function of receiving an input signal from the console 40 or an input interface 43 attached to the gantry 10 and controlling the operation of the gantry 10 and the bed 30. For example, the controller 15 receives input signals so as to control: rotation of the rotating frame 13; tilting of the gantry 10; and the operation of the bed 30 and the table 33. The control of tilting the gantry 10 is achieved by the controller 15 that rotates the rotating frame 13 around an axis parallel to the X-axis direction in accordance with tilt angle information to be inputted through the input interface 43 attached to the gantry 10. The controller 15 may be provided in the gantry 10 or in the console 40. The input interface 43 will be described below.

The bed 30 includes a base 31, a support frame 32, a table 33, and a bed driver 34. The base 31 is installed on the floor surface. The base 31 is a housing configured to support the support frame 32 in such a manner that the support frame 32 can move in the direction perpendicular to the floor surface (i.e., in the Y-axis direction). The support frame 32 is a frame provided on the upper portion of the base 31. The support frame 32 supports the table 33 in such a manner that the table 32 can slide along the rotation axis (i.e., along the Z-axis). The table 33 is a flexible plate on which the object P is placed.

The bed driver 34 is housed in the housing of the bed 30. The bed driver 34 is a motor or an actuator configured to generate power for moving the support frame 32 and the table 33 on which the object P is placed. The bed driver 34 operates under the control of the console 40, for example.

The console 40 includes a memory 41, a display 42, the input interface 43, and the processing circuitry 44. Data communication between the memory 41, the display 42, the input interface 43, and the processing circuitry 44 is performed via a bus. Although the console 40 will be described as a component separated from the gantry 10, the gantry 10 may include the entirety of the console 40 or some components of the console 40.

The memory 41 is a storage device such as a hard disk drive (HDD), a solid state drive (SSD), and an integrated circuit storage device, all of which are configured to store various information items. The memory 41 may be a portable storage medium such as a compact disc (CD), a digital versatile disc (DVD), a Blu-ray (registered trademark) disc (BD), and a flash memory, in addition to the HDD and the SSD. The memory 41 may be a drive device that reads out and writes various information items from/on a semiconductor memory element such as a flash memory and a RAM, for example. The storage region of the memory 41 may be provided in the X-ray CT apparatus 1 or in an external storage device connected via a network. The memory 41 stores the projection data and reconstructed image data, for example.

The display 42 displays various information items. For example, the display 42 displays CT images generated by the processing circuitry 44, a GUI (Graphical User Interface) for receiving various operations from a user. Various arbitrary displays can be used as the display 42 as appropriate. For example, the display 42 may be a liquid crystal display (LCD), a cathode ray tube (CRT) display, an organic electroluminescence display (OELD), or a plasma display.

The display 42 may be provided anywhere in the control room. The display 42 may also be provided in the gantry 10. The display 42 may be a desktop type or may be configured as a tablet terminal capable of wireless communication with the main body of the console 40. In addition, one or more projectors may be used as the display 42.

The input interface 43 receives various input operations from the user, converts the received input operations into electrical signals, and outputs the electrical signals to the processing circuitry 44. For example, the input interface 43 receives: acquisition conditions for acquiring projection data; reconstruction conditions for reconstructing CT images; and image processing conditions for generating post-processed images from the CT images, from the user. As the input interface 43, for example, a mouse, a keyboard, a trackball, a switch, a button, a joystick, a touchpad, and a touch panel display can be used as appropriate.

In the present embodiment, the input interface 43 is not limited to those having physical operating parts such as a mouse, a keyboard, a trackball, a switch, a button, a joystick, a touchpad, and a touch panel display. For example, aspects of the input interface 43 also include processing circuitry that receives electrical signals corresponding to input operations from an external input device provided separately from the apparatus and outputs these electrical signals to the processing circuitry 44. The input interface 43 may be provided in the gantry 10. The input interface 43 may be configured as a tablet terminal that can wirelessly communicate with the main body of the console 40, for example.

The processing circuitry 44 controls the operation of the entirety of the X-ray CT apparatus 1 in response to the electrical signals of the input operations outputted from the input interface 43. The processing circuitry 44 generates image data on the basis of the electrical signals outputted from the X-ray detector 12. For example, the processing circuitry 44 includes: a processor such as a CPU, an MPU, and a GPU; and a memory such as a ROM and a RAM, as its hardware resources. The processing circuitry 44 implements at least an imaging control function 441, a reconstruction function 442, an image processing function 443, an imaging-condition setting function 444, a tube-current command-value update function 445, and a display control function 446 by using its processor that executes the programs developed in the memory.

The respective functions 441 to 446 are not limited to an aspect of being implemented by single processing circuitry. The respective functions 441 to 446 may be implemented by: constituting the processing circuitry by combining a plurality of independent processors; and causing the respective processors to execute the programs.

The imaging control function 441 includes functions of: controlling the X-ray high-voltage device 14, the controller 15, and the DAS 18 in accordance with the imaging conditions selected or determined by the imaging-condition setting function 444; and performing X-ray CT imaging. In the present embodiment, X-ray CT imaging is performed by alternately switching the tube voltage between a first tube-voltage value and a second tube-voltage value while modulating the tube current depending on an X-ray tube angle (hereinafter, referred to as “the spectral scan method”). There is no particular restriction on the magnitude relationship between the first tube-voltage value and the second tube-voltage value. For example, it is assumed that the first tube-voltage value is a high tube-voltage value and the second tube-voltage value is a low tube-voltage value lower than the high tube-voltage value.

The reconstruction function 442 includes a function of preprocessing the projection data outputted from the DAS 18, such as logarithmic transformation processing, offset correction processing, inter-channel sensitivity correction processing, and beam hardening correction processing. The reconstruction function 442 performs reconstruction processing on the preprocessed projection data by using an appropriate method such as filtered back-projection, sequential approximate reconstruction, and machine learning so as to generate CT images.

The image processing function 443 includes a function of converting the CT images generated by the reconstruction function 442 into cross-sectional images of an arbitrary cross-section and/or rendering images from an arbitrary viewpoint. The conversion by the image processing function 443 is performed on the basis of input operations received from the user via the input interface 43. For example, the image processing function 443 performs three-dimensional image processing such as volume rendering, surface volume rendering, image-value projection processing, MPR (Multi-Planer Reconstruction) processing, and CPR (Curved MPR) processing on the CT image data so as to generate rendering images from an arbitrary viewpoint. Note that the generation of the rendering images from an arbitrary viewpoint may be directly performed by the reconstruction function 442.

The imaging-condition setting function 444 includes a function of setting imaging conditions related to a spectral scan. The imaging-condition setting function 444 selects the first tube-voltage value and the second tube-voltage value to be used in the spectral scan. The tube-voltage value is selected either manually by the user via the input interface 43 or automatically on the basis of a predetermined algorithm. The imaging-condition setting function 444 sets a tube-current table, which shows the temporal change in the tube-current value under the first tube-voltage value. The tube-current table is set either manually by the user via the input interface 43 or automatically on the basis of a predetermined algorithm.

The tube-current command-value update function 445 includes a function of updating the tube-current command-value in the table stored in a memory 148 of the X-ray high-voltage device 14 at a predetermined timing according to switching of the tube voltage, when the timing at which the tube voltage switches from a high voltage value to a low voltage value is detected. The predetermined timing is, for example, a timing of switching of the tube voltage. The predetermined timing may be a timing after a lapse of a predetermined time from the timing. The tube-current command-value update function 445 receives the spectral signal from the controller 15 and detects the timing at which the tube voltage is switched. The tube-current command-value update function 445 may receive a tube-voltage command-signal or a tube-voltage detection-signal from the X-ray high-voltage device 14 so as to detect the timing at which the tube voltage is switched.

The display control function 446 includes a function of displaying various images generated by the image processing function 443 on the display 42. The display control function 446 causes the display 42 to display a CT image, a cross-sectional image of an arbitrary cross-section, a rendering image from an arbitrary viewpoint, and a setting screen for imaging conditions, for example.

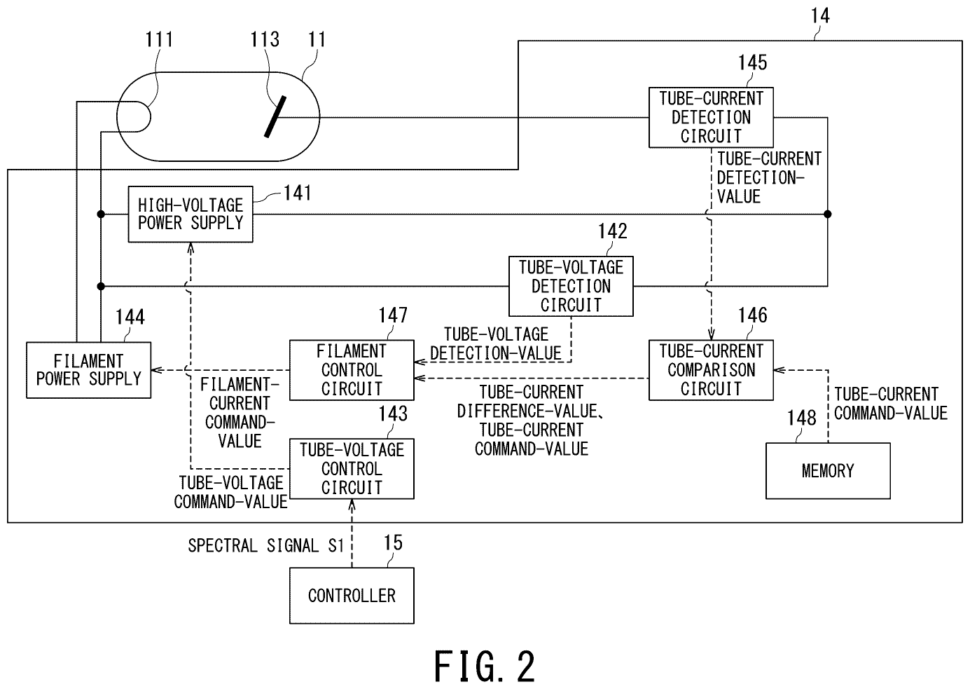

The X-ray generation system according to the present embodiment will be described in more detail by referring to the drawings. FIG. 2 is a block diagram illustrating a configuration of the X-ray generation system including the X-ray tube 11 and the X-ray high-voltage device 14.

As shown in FIG. 2, the X-ray tube 11 accommodates a cathode 111 and an anode 113. The cathode 111 has a filament made of metal such as tungsten and nickel. The cathode 111 is connected to the X-ray high-voltage device 14 via a cable, for example. When the cathode voltage is applied to the cathode 111 and the filament current is supplied to the cathode 111 from the X-ray high-voltage device 14, the cathode 111 generates heat and emits thermal electrons.

The anode 113 is a disk-shaped electrode made of heavy metal such as tungsten and molybdenum. The anode 113 rotates along with rotation of a rotor (not shown) around its axis. The tube voltage is applied as a high voltage between the cathode 111 and the anode 113 by the X-ray high-voltage device 14. The thermal electrons emitted from the cathode 111 collide with the anode 113 due to the action of the tube voltage. The anode 113 receives the thermal electrons from the cathode 111 and thereby generates X-rays.

As shown in FIG. 2, the X-ray high-voltage device 14 includes a high-voltage power supply 141, a tube-voltage detection circuit 142, a tube-voltage control circuit 143, a filament power supply 144, a tube-current detection circuit 145, a tube-current comparison circuit 146, a filament control circuit 147, and a memory 148. Each circuit of the X-ray high-voltage device 14 is realized by an ASIC and/or an FPGA, for example.

The high-voltage power supply 141 generates a high DC voltage to be applied to the X-ray tube 11 under the control of the tube-voltage control circuit 143. The high DC voltage is applied as the tube voltage between the cathode 111 and the anode 113 of the X-ray tube 11. The high-voltage power supply 141 applies the tube voltage to the X-ray tube 11 by periodically switching the tube voltage between a high tube-voltage value and a low tube-voltage value that is lower than the high tube-voltage value. The high-voltage power supply 141 is one example of an X-ray tube power supply. The high tube-voltage value is one example of a first tube-voltage value. The low tube-voltage value is one example of a second tube-voltage value.

The tube-voltage detection circuit 142 detects the voltage applied between the cathode 111 and the anode 113 as the tube voltage. The detected value of this tube-voltage is hereinafter referred to as “the tube-voltage detection-value”, the signal indicating this tube-voltage detection-value is hereinafter referred to as “the tube-voltage detection-signal”, and this tube-voltage detection-signal is transmitted to the filament control circuit 147.

In the spectral scan method, the tube-voltage control circuit 143 switches the tube voltage to be applied to the X-ray tube 11 between the high tube-voltage value (i.e., the first tube-voltage value) and the low tube-voltage value (i.e., the second tube-voltage value). Specifically, the tube-voltage control circuit 143 receives the spectral signal S1 (i.e., the tube-voltage modulation signal), which is a control signal for commanding the timing of switching the tube voltage from the controller 15. The tube-voltage control circuit 143 switches the tube-voltage command-value on the basis of the timing at which the spectral signal S1 is switched. The tube-voltage control circuit 143 controls the tube voltage by transmitting the tube-voltage command-signal indicating the tube-voltage command-value to the high-voltage power supply 141.

The filament power supply 144 generates the filament current for heating up the filament of the cathode 111 under the control of the filament control circuit 147. In detail, the filament power supply 144 has an inverter circuit that controls the voltage to be applied to the filament of the cathode 111. The filament power supply 144 generates the filament current by applying the voltage to the filament on the basis of a filament-current command-value specified by the filament control circuit 147.

The tube-current detection circuit 145 is connected between the high-voltage power supply 141 and the X-ray tube 11. The tube-current detection circuit 145 detects the electric current, which flows due to the flow of the thermal electrons from the cathode 111 to the anode 113, as the tube current. The detected value of this tube-current value is hereinafter referred to as “the tube-current detection-value”, the signal indicating this tube-current detection-value is hereinafter referred to as “the tube-current detection-signal”, and this tube-current detection-signal is transmitted to the tube-current comparison circuit 146.

The tube-current comparison circuit 146 receives the tube-current command signal indicating the command value of the tube current from the memory 148 (hereinafter referred to as “the tube-current command-value”), and also receives the tube-current detection-signal from the tube-current detection circuit 145. The tube-current comparison circuit 146 generates a difference current signal that indicates the difference between the tube-current command-value indicated by the tube-current command-signal and the tube-current detection-value indicated by the tube-current detection-signal, and this difference between both is hereinafter referred to as “the tube-current difference-value”. The tube-current comparison circuit 146 transmits the tube-current command-signal and the difference current signal to the filament control circuit 147. The tube-current comparison circuit 146 may transmit the tube-current detection-signal indicating the tube-current detection-value to the filament control circuit 147.

The filament control circuit 147 controls the tube current by controlling the filament current generated by the filament power supply 144. The filament control circuit 147 receives the tube-voltage detection-signal from the tube-voltage detection circuit 142 during each scan. The filament control circuit 147 receives the difference current signal and the tube-current command-signal from the tube-current comparison circuit 146 during each scan. The filament control circuit 147 specifies the filament-current command-value corresponding to the command value of the tube current at a predetermined tube voltage on the basis of characteristic data. The characteristic data will be described below.

When the tube-voltage detection-value indicated by the tube-voltage detection-signal is the high tube-voltage value, the filament control circuit 147 performs tube-current feedback-control. In other words, the filament control circuit 147 determines the filament-current command-value in such a manner that the tube-current detection-value converges to the tube-current command-value as quickly as possible. In detail, the filament control circuit 147 receives the difference current signal from the tube-current comparison circuit 146, and calculates the filament-current command-value from the tube-current difference-value indicated by the difference current signal on the basis of general feedback control such as PID (Proportional Integral Derivative) control. The filament control circuit 147 then transmits the filament-current command-signal indicating the filament-current command-value to the filament power supply 144.

In other words, on the basis of the characteristic data, the filament control circuit 147 specifies the filament-current command-value in the high tube-voltage period from both the tube-current command-value updated by the tube-current command-value update function 445 and the tube-current detection-value detected by the tube-current detection circuit 145. The filament power supply 144 applies the voltage to the filament during the high tube-voltage period on the basis of the specified filament-current command-value.

When the tube-voltage detection-value indicated by the tube-voltage detection-signal is the low tube-voltage, the filament control circuit 147 performs filament-current feedback-control. In other words, the filament control circuit 147 determines a filament-current setting-value in such a manner that the filament-current detection-value converges to the filament-current command-value as quickly as possible. Specifically, the filament control circuit 147 receives the tube-current command-signal from the tube-current comparison circuit 146, and calculates the filament-current command-value in the high tube-voltage period from the tube-current command-value indicated by the tube-current command-signal. The details will be described below. The filament control circuit 147 then transmits the filament-current command-signal indicating the filament-current command-value to the filament power supply 144. Note that the filament control circuit 147 may read out the tube-current command-value from the table in the memory 148.

The memory 148 stores various control parameters, such as the tube-current command-value, predetermined thresholds (convergence criteria) to be used in various feedback control methods, for example. In detail, for each tube voltage to be applied to the X-ray tube 11, the memory 148 stores: (i) the characteristic data in which the filament current flowing through the filament in the X-ray tube 11 and the tube current flowing through the X-ray tube 11 are associated with each other; and (ii) the command value of the tube current. The memory 148 is a storage device such as a HDD, an SSD, and an integrated circuit storage device, all of which store various information items. The memory 148 may be a drive device that reads out and writes various information items from/on a CD, DVD, and a semiconductor memory element such as a flash memory and a RAM. The storage region of the memory 148 may be provided in the X-ray high-voltage device 14 or in an external storage device connected via a network.

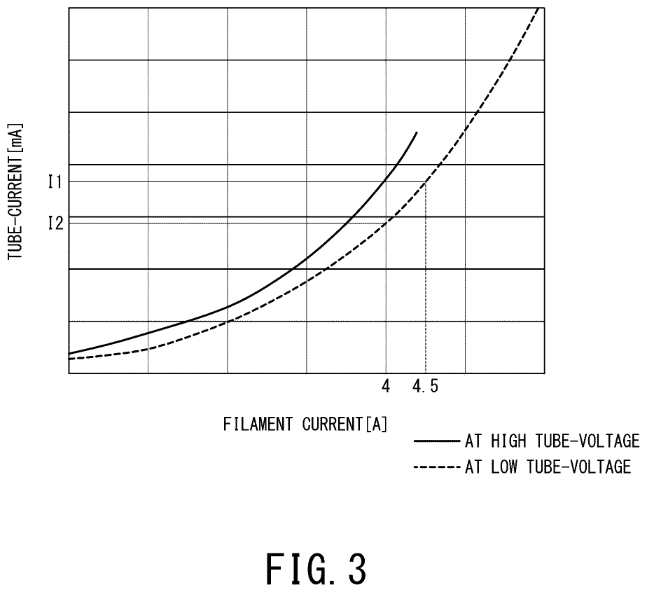

FIG. 3 is a graph illustrating characteristics between the filament current and the tube current according to the first embodiment. Of the two curves in the graph, one shows the characteristics at the high tube-voltage, and the other shows the characteristics at the low tube-voltage. These graph data are stored in the memory 148 of the X-ray high-voltage device 14, for example. The filament control circuit 147 refers to the graph data in the memory 148 when calculating the filament-current command-value during the high tube-voltage period from the tube-current command-value. In other words, the filament control circuit 147 specifies the filament-current command-value corresponding to the tube-current command-value from the graph data at the high tube-voltage. Note that the filament control circuit 147 may refer to a lookup table of discrete values instead of the graph of continuous values.

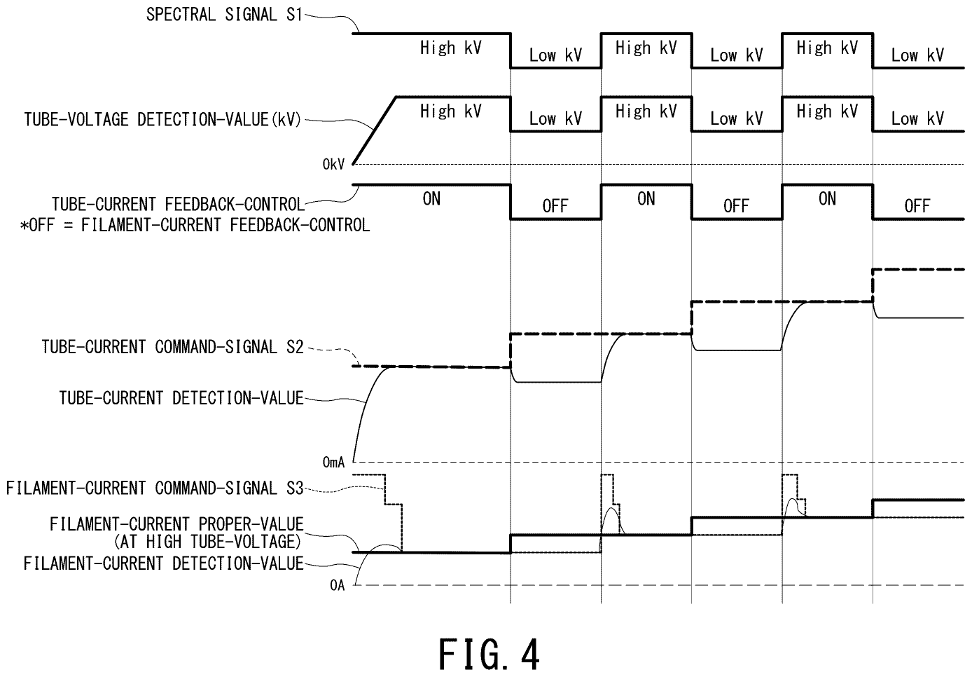

FIG. 4 is a timing chart illustrating temporal changes in each signal and each detection value according to the first embodiment. FIG. 4 shows the timing chart for updating the tube-current command-signal S2 when the spectral signal S1 switches from the high tube-voltage (High kV) to the low tube-voltage (low kV). The details will be described below.

The spectral signal S1 is a control signal that commands the timing of switching the tube voltage. The spectral signal S1 is transmitted from the controller 15 to the tube-voltage control circuit 143 of the X-ray high-voltage device 14. As shown in FIG. 4, the spectral signal S1 periodically and alternately switches between the high tube-voltage and the low tube-voltage.

The tube voltage is a voltage to be applied to the X-ray tube 11 from the high-voltage power supply 141 under the control of the tube-voltage control circuit 143 that controls the high-voltage power supply 141 in accordance with the spectral signal S1. The tube-voltage detection-value is the value of the tube voltage detected by the tube-voltage detection circuit 142. The tube-voltage detection-value is alternately switched between the high tube-voltage and the low tube-voltage, similarly to the spectral signal S1. The tube-voltage detection-value does not rise immediately at the time of starting from 0 kV, and gradually increases toward the high tube-voltage value.

The tube-current feedback-control is turned on (i.e., executed) during the high tube-voltage period. The tube-current feedback-control is turned off (i.e., not executed) during the low tube-voltage period. During the low tube-voltage period, the filament-current feedback-control is executed instead of the tube-current feedback-control.

The tube-current command-signal S2 is a signal that indicates the command value of the tube current. The tube-current command-signal S2 is transmitted to the filament control circuit 147 after the tube-current command-value having been read out from the memory 148 by the tube-current comparison circuit 146 is set. The tube-current command-value update function 445 updates the tube-current command-value in the memory 148 at the timing at which the tube voltage switches from the high tube-voltage value to the low tube-voltage value. As a result of this update, the tube-current command-signal S2 is updated at the timing at which the tube voltage switches from the high tube-voltage value to the low tube-voltage value.

The tube-current detection-value is the value of the tube current detected by the tube-current detection circuit 145. The tube current of the X-ray tube 11 has characteristics that depend on the filament current and the tube voltage. Since the tube-current detection-value during the high tube-voltage period is initially much lower than the tube-current command-value, it takes time for the tube-current detection-value to converge to the tube-current command-value. During the low tube-voltage period, the tube-voltage detection-value is low despite the constant filament-current detection-value, so the tube-current detection-value initially decreases and then stabilizes.

The filament-current command-signal S3 is a signal that indicates the filament-current command-value. The filament-current command-value is specified based on the tube-current difference-value and/or the filament-current proper-value. The filament-current command-signal S3 is transmitted from the filament control circuit 147 to the filament power supply 144. As shown in FIG. 4, during the high tube-voltage period, the tube-current feedback-control is turned on, so the graph of the filament-current command-signal S3 becomes highly projected in order to reduce the initially large tube-current difference-value. When the tube-current difference-value is reduced and the tube-current detection-value converges to the tube-current command-value, the filament-current command-signal S3 becomes stable. During the low tube-voltage period, because the tube-current feedback-control is turned off and the filament-current feedback-control is turned on, the filament-current command signal S3 remains stable.

The filament-current detection-value is the value of the filament current detected by the filament power supply 144. Depending on the filament-current command-signal S3, the filament-current detection-value spikes at the beginning of the high tube-voltage period but remains stable for the rest of the period.

According to the above-described configuration, the tube-current command-signal S2 is updated at the time at which the spectral signal S1, the tube-voltage command-value, or the tube-voltage detection-value switches from the high tube-voltage to the low tube-voltage. Since the tube-current command-signal S2 is not updated during the high tube-voltage period in the above-described configuration, the filament current remains stable even if it transitions from the high tube-voltage period to the low tube-voltage period.

In the control shown in the timing chart of FIG. 4, during the low tube-voltage period, the filament control circuit 147 specifies the filament-current command-value in the high tube-voltage period from the tube-current command-value, and transmits the filament-current command-signal indicating this command value to the filament power supply 144. For example, according to FIG. 3, when the tube-current command-value is I1, the filament current is controlled to flow only at 4.5 A in the low tube-voltage characteristics. If it transitions from the low tube-voltage period to the high tube-voltage period under this state, the filament current is larger than 4 A under the high tube-voltage, so the tube current temporarily becomes larger than the command value I1.

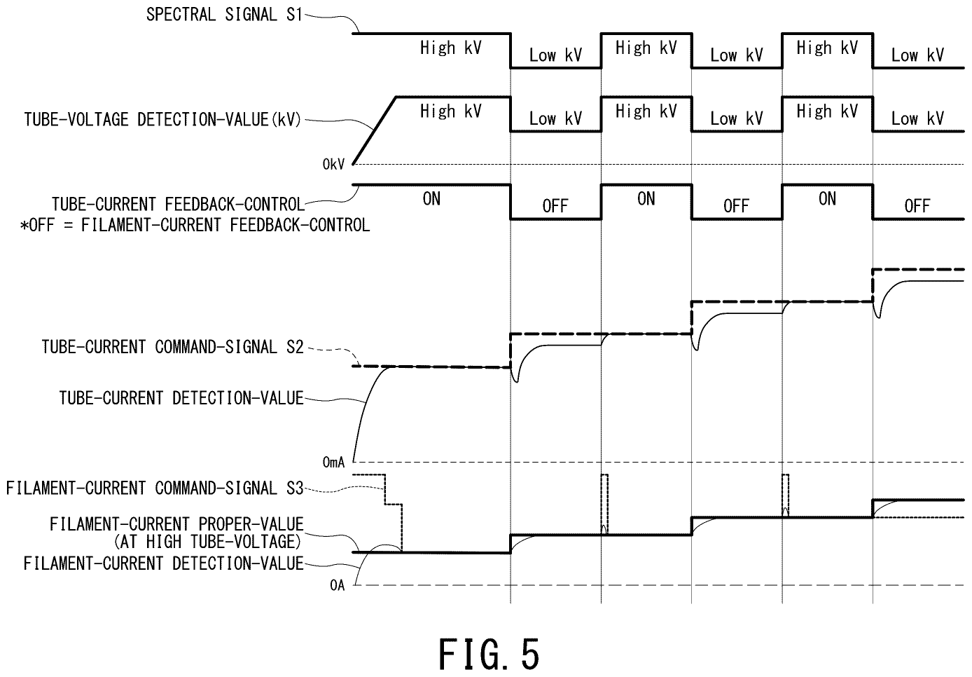

Accordingly, in the control of FIG. 5, during the low tube-voltage period, the filament control circuit 147 specifies 4 A, which is the filament current during the high tube-voltage period, as the command value from the tube-current command value I1. FIG. 5 is another timing chart illustrating temporal changes in each signal and each detection value according to the first embodiment. FIG. 5 shows a timing chart of the case where the filament-current command-value in the high tube-voltage period is specified from the tube-current command-value and the filament-current feedback-control is performed in addition to the control shown in FIG. 4 during the low tube-voltage period. In this case, the filament control circuit 147 specifies the filament-current command-value in the high tube-voltage period from the tube-current command-value updated by the tube-current command-value update function 445 on the basis of the characteristic data. The filament power supply 144 then applies the voltage to the filament during the low tube-voltage period on the basis of the filament-current command-value specified by the filament control circuit 147. Hereinbelow, a description will be mainly given for the differences from FIG. 4.

The spectral signal S1 and the tube-voltage detection-value are the same as in FIG. 4. The tube-current feedback-control is turned on during the high tube-voltage period and is turned off during the low tube-voltage period. During the low tube-voltage period, the filament-current feedback-control is executed instead of the tube-current feedback-control. The tube-current command-signal S2 is updated at the timing at which the tube voltage switches from the high voltage value to the low voltage value, similarly to FIG. 4.

The tube-current detection-value is 0 milliamperes at the beginning of the first high tube-voltage period, and this value of 0 mA is much lower than the tube-current command-value, so it takes time to converge to the tube-current command-value. At the timing at which it switches from the high tube-voltage period to the low tube-voltage period, the tube-current command signal S2 is updated and the filament-current feedback-control is performed in such a manner that the filament-current command-value at the high tube-voltage calculated from the tube-current command-value is used as the proper value. During the low tube-voltage period, though the tube-current detection-value initially drops similarly to FIG. 4, the tube-current detection-value then stabilizes near the tube-current command-value. Thus, after the second high tube-voltage period, the tube-current detection-value quickly converges to the tube-current command-value.

As shown in FIG. 5, during the high tube-voltage period, the tube-current feedback-control is turned on, and the graph of the filament-current command-signal S3 becomes highly projected in order to reduce the initially large tube-current difference-value. When the tube-current difference-value is reduced and the tube-current detection-value converges to the tube-current command-value, the filament-current command-signal S3 becomes stable. During the low tube-voltage period, the filament-current feedback-control is performed, and the filament-current command-signal S3 indicates the filament-current command-value at the high tube-voltage as the proper value.

In response to the filament-current command signal S3, the filament-current detection value spikes at the beginning of the high tube-voltage period but then remains stable for the rest of the period. The filament-current detection-value first rises gradually to the proper value during the low tube-voltage period, and then remains stable.

According to the above-described configuration, the filament control circuit 147 specifies the filament-current command-value at the high tube-voltage from the updated tube-current command-value during the low tube-voltage period, and performs feedback control with respect to the filament-current command-value, and thereby, the filament current is stabilized. Under this control, the necessary filament current is already flowing at the timing of transitioning to the next high tube-voltage period, and thus, the tube-current detection-value quickly converges to the tube-current command-value. Hence, during the high tube-voltage period, the actual tube current quickly converges to the command value, this enables stable control of the tube current and contributes to improvement in quality of X-ray images.

In the filament-current feedback control shown in FIG. 5, as shown in FIG. 3, the filament current during the high tube-voltage period for the tube-current command-value I1 is 4 A, so the tube current during the low tube-voltage period is I2, which is smaller than the tube-current command-value I1. Thus, as a modification of the first embodiment, the filament control circuit 147 may control the filament current in such a manner that the tube-current detection-value approaches the tube-current command-value I1 as closely as possible during the low tube-voltage period. For example, the filament control circuit 147 may set the filament-current value during the low tube-voltage period with respect to the tube-current value between I1 and I2 as the filament-current command-value.

The details will be described below by referring to FIG. 3. On the basis of the characteristic data, the filament control circuit 147 specifies the filament current value 4 A during the high tube-voltage period from the tube-current command-value I1 updated by the tube-current command-value update function 445. Next, the filament control circuit 147 specifies the tube-current value I2 during the low tube-voltage period from the filament current value 4 A. The filament control circuit 147 then specifies the filament-current command-value (i.e., intermediate value between 4 A and 4.5 A) during the low tube-voltage period from the intermediate value between the tube-current command-value I1 and the tube-current value I2. The filament power supply 144 applies the voltage to the filament during the low tube-voltage period on the basis of the specified filament-current command-value.

### Second Embodiment

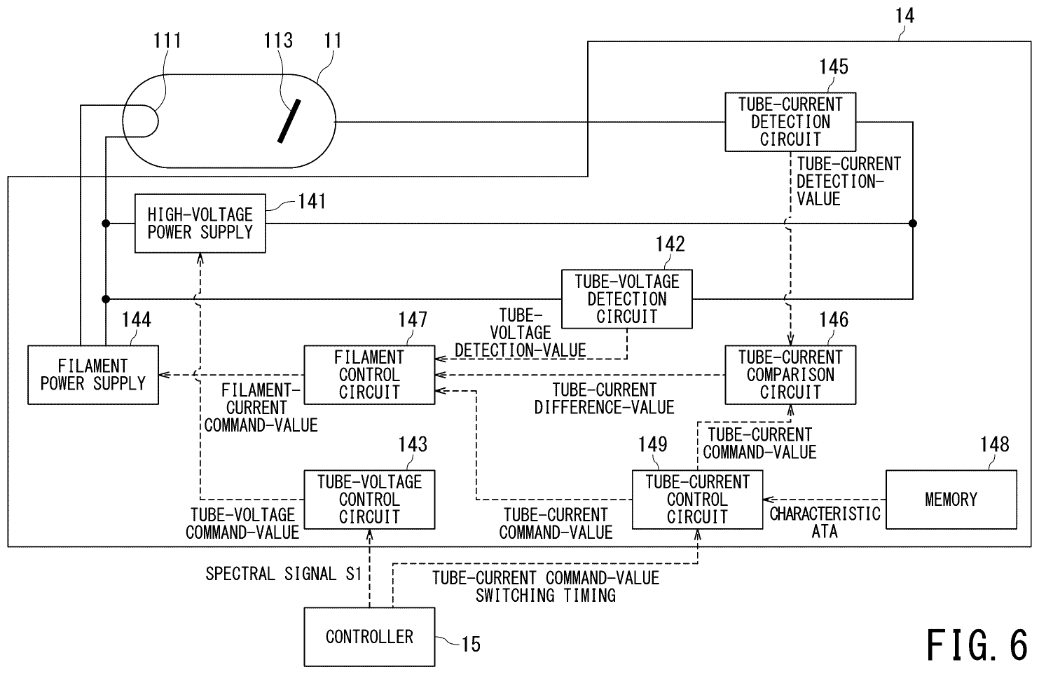

An X-ray generation system according to a second embodiment will be described in detail with reference to the drawings. The configuration of the X-ray CT apparatus 1 shown in FIG. 1 is applicable to the second embodiment. FIG. 6 is a block diagram illustrating a configuration of an X-ray generation system including the X-ray tube 11 and the X-ray high-voltage device 14 in FIG. 1. In the following, differences from FIG. 2 in the first embodiment will be described.

As shown in FIG. 6, the X-ray high-voltage device 14 includes the high-voltage power supply 141, the tube-voltage detection circuit 142, the tube-voltage control circuit 143, the filament power supply 144, the tube-current detection circuit 145, the tube-current comparison circuit 146, the filament control circuit 147, the memory 148, and a tube-current control circuit 149. Each circuit of the X-ray high-voltage device 14 is realized by an ASIC and/or an FPGA, for example.

The high-voltage power supply 141, the tube-voltage detection circuit 142, the tube-voltage control circuit 143, the filament power supply 144, and the tube-current detection circuit 145 are the same as those described with reference to FIG. 2.

The tube-current comparison circuit 146 receives the tube-current command-signal indicating the tube-current command-value from the tube-current control circuit 149, and the tube-current detection-signal from the tube-current detection circuit 145. The tube-current comparison circuit 146 generates the difference current signal that indicates the tube-current difference-value between the tube-current command-value indicated by the tube-current command-signal and the tube-current detection-value indicated by the tube-current detection-signal. The tube-current comparison circuit 146 transmits the difference current signal to the filament control circuit 147.

The filament control circuit 147 controls the tube current by controlling the filament current generated by the filament power supply 144. The filament control circuit 147 receives the tube-voltage detection-signal from the tube-voltage detection circuit 142 during scanning. Furthermore, the filament control circuit 147 receives the tube-current command-signal from the tube-current control circuit 149 during scanning, and receives the difference current signal from the tube-current comparison circuit 146. The filament control circuit 147 specifies the filament-current command-value corresponding to the tube-current command-value at a predetermined tube voltage on the basis of the characteristic data. The characteristic data indicates characteristics between the filament current and the tube current for each tube voltage.

When the tube-voltage detection-value indicated by the tube-voltage detection-signal is the high tube-voltage value, the filament control circuit 147 specifies the filament-current command-value in the high tube-voltage period from the tube-current command-value on the basis of the characteristic data. The filament power supply 144 applies the voltage to the filament during the high tube-voltage period on the basis of the specified filament-current command-value.

When the tube-voltage detection-value indicated by the tube-voltage detection-signal is the low tube-voltage value, the filament control circuit 147 specifies the filament-current command-value in the low tube-voltage period from the tube-current command-value on the basis of the characteristic data. The filament power supply 144 applies the voltage to the filament during the low tube-voltage period on the basis of the specified filament-current command-value.

The filament control circuit 147 performs tube-current feedback-control. In other words, the filament control circuit 147 determines the filament-current command-value in such a manner that the tube-current detection-value converges to the tube-current command-value as quickly as possible. In detail, the filament control circuit 147 receives the difference current signal from the tube-current comparison circuit 146, and calculates the filament-current command-value from the tube-current difference-value indicated by the difference current signal on the basis of general feedback control such as PID (Proportional Integral Derivative) control. The filament control circuit 147 then transmits the filament-current command-signal indicating the filament-current command-value to the filament power supply 144. The filament power supply 144 applies the voltage to the filament on the basis of the filament-current command-signal received from the filament control circuit 147.

The memory 148 stores various control parameters, such as the tube-current command-value, and predetermined thresholds (convergence criteria) to be used in various feedback control methods, for example. In detail, for each tube voltage to be applied to the X-ray tube 11, the memory 148 stores the characteristic data in which the filament current flowing through the filament in the X-ray tube 11 and the tube current flowing through the X-ray tube 11 are associated with each other. The memory 148 is a storage device such as a HDD, an SSD, and an integrated circuit storage device, all of which store various information items. The memory 148 may be a drive device that reads out and writes various information items from/on a CD, a DVD, a BD, and a semiconductor memory element such as a flash memory and a RAM. The storage region of the memory 148 may be provided in the X-ray high-voltage device 14 or in an external storage device connected via a network.

The tube-current control circuit 149 determines the tube-current command-value. In detail, in a period of the high tube-voltage value, the tube-current control circuit 149 acquires a predetermined tube-current value from the controller 15, and determines the same to be the tube-current command-value. In a period of the low tube-voltage value, the tube-current control circuit 149 specifies the filament current value associated with the predetermined tube-current value corresponding to the high tube-voltage value on the basis of the characteristic data stored in the memory 148, and determines the tube-current value associated with the specified filament current value corresponding to the low tube-voltage value to be the tube-current command-value. The tube-current control circuit 149 then transmits the tube-current command-signal indicating the determined tube-current command-value to the tube-current comparison circuit 146 and the filament control circuit 147. A transmission timing of the tube-current command-signal will be described later. The tube-current control circuit 149 is an example of processing circuitry.

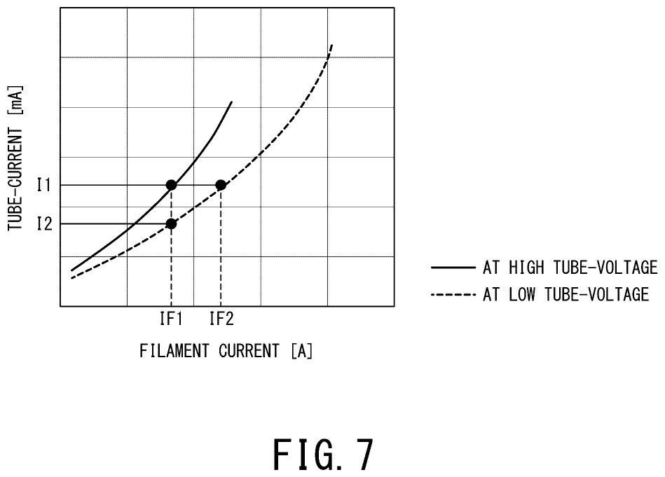

FIG. 7 is a graph illustrating characteristics between the filament current and the tube current according to the second embodiment. The graph is an example of the characteristic data. Of the two curves in the graph, a solid line indicates the characteristics at the high tube-voltage, and a dashed line indicates the characteristics at the low tube-voltage. These graph data are stored in the memory 148 of the X-ray high-voltage device 14, for example.

An example operation at the time when the tube-current control circuit 149 determines I1 as the tube-current command-value at the time of high tube-voltage will be described with reference to FIG. 7. When the tube-current value converges to I1 during the high tube-voltage period, the filament current value IF1 flows. In views of a first half after the high tube-voltage period transitions to the low tube-voltage period, the tube-current control circuit 149 maintains the tube-current command-value at I1 in the high tube-voltage period. When the tube-current value converges to I1 during the low tube-voltage period, the filament current value IF2 flows. In views of a second half in the low tube-voltage period, the tube-current control circuit 149 switches the tube-current command-value to I2. When the tube-current value converges to I2 during the low tube-voltage period, the filament current value IF1 flows. After the low tube-voltage period transitions to the high tube-voltage period, the tube-current control circuit 149 returns the tube-current command-value to I1. Accordingly, even when the tube-current command-value is increased from I2 to I1, because IF1 is already flowing as the filament current, the tube-current value does not go over I1 and is converged.

Then, when calculating the filament-current command-value from the tube-current command-value determined by the tube-current control circuit 149, the filament control circuit 147 refers to data of the graph in the memory 148. In other words, the filament control circuit 147 specifies the filament-current command-value corresponding to the tube-current command-value from the graph data at the high tube-voltage or the low tube-voltage. Note that the filament control circuit 147 may refer to a lookup table of discrete values instead of the graph of continuous values.

Note that in the graph in FIG. 7, control may be performed such that a difference between the tube currents I1 and I2 is reduced. For example, the filament control circuit 147 specifies the filament current value IF1 that is associated with the tube-current value I1 corresponding to the high tube-voltage value on the basis of the characteristic data. The filament control circuit 147 then specifies the tube-current value I2 that is associated with the specified filament current value IF1 corresponding to the low tube-voltage value. The filament control circuit 147 then specifies a filament current value (an intermediate value between IF1 and IF2) corresponding to the low tube-voltage value and associated with an intermediate value between the tube-current value I1 and the specified tube-current value I2.

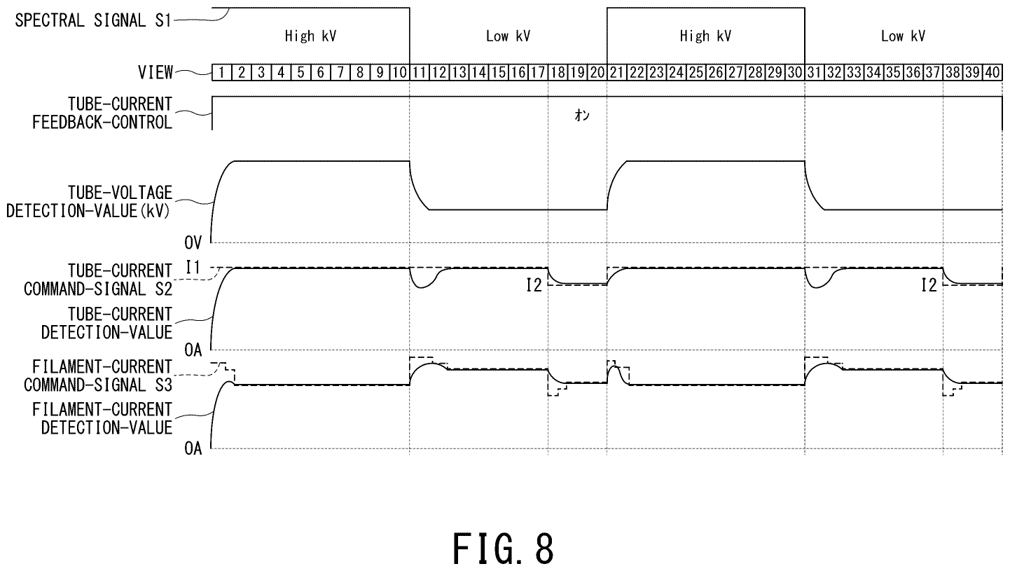

FIG. 8 is a timing chart illustrating temporal changes in each signal and each detection value according to the second embodiment. FIG. 8 shows a timing chart for updating the tube-current command-signal S2 at a timing of switching of the view. Details will be given below.

The spectral signal S1 is a control signal that commands the timing of switching the tube voltage. The spectral signal S1 is transmitted from the controller 15 to the tube-voltage control circuit 143 and the tube-current control circuit 149 of the X-ray high-voltage device 14. As shown in FIG. 8, the spectral signal S1 periodically and alternately switches between the high tube-voltage and the low tube-voltage.

One view period is determined by the number of times of data acquisition per rotation of the rotating frame 13. In the example shown in FIG. 8, the spectral signal S1 is switched every 10 views. The controller 15 notifies the tube-current control circuit 149 of the timing when the view switches. The tube-current control circuit 149 acquires the timing of switching of the view from the controller 15, and switches the tube-current command-value at a predetermined timing according to the timing of switching of the view. The predetermined timing includes the timing when the view switches. The controller 15 and the tube-current control circuit 149 are examples of processing circuitry.

The tube-current feedback-control is on at all times, and is continuously performed by the tube-current comparison circuit 146 and the filament control circuit 147.

The tube voltage is a voltage to be applied to the X-ray tube 11 from the high-voltage power supply 141 under the control of the tube-voltage control circuit 143 that controls the high-voltage power supply 141 in accordance with the spectral signal S1. The tube-voltage detection-value is the value of the tube voltage detected by the tube-voltage detection circuit 142. The tube-voltage detection-value is alternately switched between the high tube-voltage and the low tube-voltage, similarly to the spectral signal S1. The tube-voltage detection-value does not rise immediately at the time of starting from 0 V, and gradually increases toward the high tube-voltage value.

The tube-current command-signal S2 is a signal that indicates the tube-current command-value. The tube-current command-signal S2 is transmitted to the tube-current comparison circuit 146 and the filament control circuit 147 after the tube-current command-value having been determined by the tube-current control circuit 149 is set. The tube-current detection-value is the value of the tube current detected by the tube-current detection circuit 145. The tube current of the X-ray tube 11 has characteristics that depend on the filament current and the tube voltage. Switching of the tube-current command-value and change in the tube-current detection-value will be described below.

As shown in FIG. 8, the tube-current control circuit 149 sets the tube-current command-value to I1 in the high tube-voltage period and a first part (for example, 7 views) of the low tube-voltage period after spectral scan is started. When the high tube-voltage period is started, the tube-current detection-value does not rise immediately but is soon converged to I1. When the low tube-voltage period is started, the tube-current detection-value converges to I1 in a short time after a downturn.

The controller 15 specifies a timing of switching of the view when the tube-current command-value is to be switched from the tube-current value I1, on the basis of the difference between the filament current value IF1 that is associated with the tube-current value I1 corresponding to the high tube-voltage value and the filament current value IF2 that is associated with the tube-current value I1 corresponding to the low tube-voltage value, and notifies the tube-current control circuit 149 of the timing.

In the low tube-voltage period, the tube-current control circuit 149 switches the tube-current command-value from the tube-current value I1 to the tube-current value I2 that is associated with the filament current value IF1 corresponding to the low tube-voltage value, at the timing of switching of the view that is specified and notified by the controller 15. The tube-current control circuit 149 then transmits the tube-current command-signal S2 indicating the tube-current command-value I2 after switching, to the tube-current comparison circuit 146 and the filament control circuit 147.

Accordingly, by suppressing the tube current in a second part (for example, 3 views) of the low tube-voltage period from the above-described timing, overshoot of the tube current at the time of switching of the tube voltage from the low tube-voltage value to the high tube-voltage value can be avoided, accordingly unnecessary radiation exposure of a patient or a user can be prevented. Furthermore, the tube current of the X-ray tube 11 can be stabilized, and quality of CT images can be improved.

The controller 15 notifies the tube-current control circuit 149 of the timing when the tube voltage switches from the low tube-voltage value to the high tube-voltage value. The tube-current control circuit 149 returns the tube-current command-value to the tube-current value I1 at the timing notified by the controller 15, or in other words, when the tube voltage is switched from the low tube-voltage value to the high tube-voltage value. The tube-current control circuit 149 then transmits the tube-current command-signal S2 indicating the tube-current command-value I1 to the tube-current comparison circuit 146 and the filament control circuit 147.

The tube-current command-value I2 that is temporarily suppressed in the low tube-voltage period is thus raised to the original tube-current command-value I1 at the time of switching to the high tube-voltage period, and thus, rise of the tube-current detection-value is delayed. Accordingly, at least the first view after the tube voltage is switched to the high tube-voltage value possibly becomes invalid, but it is more important to prevent unnecessary radiation exposure of a patient or a user, and to stabilize the tube current of the X-ray tube 11.

The filament-current command-signal S3 is a signal that indicates the filament-current command-value. The filament-current command-value is specified based on the tube-current command-value and/or the tube-current difference-value. The filament-current command-signal S3 is transmitted from the filament control circuit 147 to the filament power supply 144. The filament power supply 144 applies the voltage to the filament on the basis of the filament-current command-signal S3 received from the filament control circuit 147. The filament-current detection-value is the value of the filament current detected by the filament power supply 144.

In the second embodiment, a description is given of a configuration where the X-ray CT apparatus 1 includes the X-ray high-voltage device 14 and the controller 15. However, the X-ray high-voltage device 14 and the controller 15 may be installed as hardware pieces separate from the X-ray CT apparatus 1, and may control the voltage and current of the X-ray tube 11 of the X-ray CT apparatus 1 from outside. In other words, a system may be configured by including the X-ray CT apparatus 1 and the controller of the X-ray CT apparatus 1.

According to at least one embodiment described above, the tube current of the X-ray tube can be stabilized in the tube current modulation scan of the X-ray CT apparatus.

While certain embodiments have been described, these embodiments have been presented by way of example only, and are not intended to limit the scope of the invention. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. An X-ray CT apparatus that can perform a tube-current modulation scan, the X-ray CT apparatus comprising:
an X-ray tube configured to irradiate an object with X-rays;
a memory configured to store, for each tube voltage to be applied to the X-ray tube, characteristic data in which a filament current flowing through a filament in the X-ray tube and the tube current are associated with each other;
an X-ray tube power supply configured to apply the tube voltage to the X-ray tube by periodically switching the tube voltage between a first tube-voltage value and a second tube-voltage value lower than the first tube-voltage value;
processing circuitry configured to update the command value of the tube current at a predetermined timing according to switching of the tube voltage; and
a filament control circuit configured to specify a filament-current command-value corresponding to the command value of the tube current based on the characteristic data.

2. The X-ray CT apparatus according to claim 1, further comprising a filament power supply configured to apply a voltage to the filament based on a specified filament-current command-value, wherein:
the processing circuitry is configured to update the command value of the tube current, as the predetermined timing, when the tube voltage switches from the first tube-voltage value to the second tube-voltage value; and
the filament control circuit is configured to specify a filament-current command-value corresponding to the command value of the tube current at a predetermined tube voltage based on the characteristic data.

3. The X-ray CT apparatus according to claim 2, wherein:
the filament control circuitry is configured to specify the filament-current command-value in a period during which the tube voltage is the first tube-voltage value from an updated command value of the tube current based on the characteristic data; and
the filament power supply is configured to apply a voltage to the filament based on the specified filament-current command-value in a period during which the tube voltage is the second tube-voltage value.

4. The X-ray CT apparatus according to claim 2, further comprising a tube-current detection circuit configured to detect the tube current, wherein:
the filament control circuit is configured to specify the filament-current command-value in a period during which the tube voltage is the first tube-voltage value from an updated command value of the tube current and a detected detection value of the tube current based on the characteristic data; and
the filament power supply is configured to apply a voltage to the filament during the period based on the specified filament-current command-value.

5. The X-ray CT apparatus according to claim 2, wherein:
the filament control circuit is configured to use the characteristic data for
specifying a value of the filament current in a period during which the tube voltage is the first tube-voltage value from the updated command value of the tube current,
specifying a value of the tube current in a period during which the tube voltage is the second tube-voltage value from a value of the filament current, and
specifying the filament-current command-value in a period during which the tube voltage is the second tube-voltage value from an intermediate value between the command value of the tube current and the value of the tube current; and
the filament power supply is configured to apply a voltage to the filament based on the specified filament-current command-value in a period during which the tube voltage is the second tube-voltage value.

6. The X-ray CT apparatus according to claim 1, wherein:
the processing circuitry is configured to update the command value of the tube current, as the predetermined timing, at a timing of switching of a view determined by the number of times of data acquisition per rotation of a rotating frame.

7. The X-ray CT apparatus according to claim 6, wherein:
the processing circuitry is configured to determine, in a period of the first tube-voltage value, a predetermined tube-current value as the command value of the tube current; and
the processing circuitry is configured to specify, in a period of the second tube-voltage value, based on the characteristic data, a filament current value that is associated with the predetermined tube-current value corresponding to the first tube-voltage value, and to determine, as the command value of the tube current, a tube-current value that is associated with the specified filament current value corresponding to the second tube-voltage value.

8. The X-ray CT apparatus according to claim 7, wherein:
the processing circuitry is configured to determine the predetermined tube-current value as the command value of the tube current, when the tube voltage is switched from the second tube-voltage value to the first tube-voltage value.

9. The X-ray CT apparatus according to claim 7, wherein:
the processing circuitry is configured to specify a timing of switching of the view when the command value of the tube current is to be switched from the predetermined tube-current value, based on a difference between the filament current value that is associated with the predetermined tube-current value corresponding to the first tube-voltage value and the filament current value that is associated with the predetermined tube-current value corresponding to the second tube-voltage value; and
the processing circuitry is configured to switch the command value of the tube current from the predetermined tube-current value to the tube-current value that is associated with the specified filament current value corresponding to the second tube-voltage value, at the specified timing of switching of the view in the period of the second tube-voltage value.

10. The X-ray CT apparatus according to claim 7, wherein:
the filament control circuit is configured to use the characteristic data for
specifying the filament current value that is associated with the predetermined tube-current value corresponding to the first tube-voltage value,
specifying the tube-current value that is associated with the specified filament current value corresponding to the second tube-voltage value, and
specifying a filament current value corresponding to the second tube-voltage value, and associated with an intermediate value of the predetermined tube-current value and the specified tube-current value.

11. A controller of an X-ray CT apparatus that can perform a tube-current modulation scan, the controller comprising:
a memory configured to store characteristic data, for each tube voltage to be applied to the X-ray tube,
the X-ray tube being configured to irradiate an object with X-rays,
the characteristic data being data in which a filament current flowing through a filament in the X-ray tube and the tube current are associated with each other;
an X-ray tube power supply configured to apply the tube voltage to the X-ray tube by periodically switching the tube voltage between a first tube-voltage value and a second tube-voltage value lower than the first tube-voltage value;
processing circuitry configured to update the command value of the tube current at a predetermined timing according to switching of the tube voltage; and
a filament control circuit configured to specify a filament-current command-value corresponding to the command value of the tube current based on the characteristic data.

12. The controller according to claim 11, further comprising a filament power supply configured to apply a voltage to the filament based on a specified filament-current command-value, wherein:
the processing circuitry is configured to update the command value of the tube current, as the predetermined timing, when the tube voltage switches from the first tube-voltage value to the second tube-voltage value; and
the filament control circuit is configured to specify a filament-current command-value corresponding to the command value of the tube current at a predetermined tube voltage based on the characteristic data.

13. The controller according to claim 11, wherein:
the processing circuitry is configured to update the command value of the tube current, as the predetermined timing, at a timing of switching of a view determined by the number of times of data acquisition per rotation of a rotating frame.

14. A control method for an X-ray CT apparatus that can perform a tube-current modulation scan, the control method comprising:
storing characteristic data, for each tube voltage to be applied to the X-ray tube,
the X-ray tube being configured to irradiate an object with X-rays,
the characteristic data being data in which a filament current flowing through a filament in the X-ray tube and the tube current are associated with each other;
applying the tube voltage to the X-ray tube by periodically switching the tube voltage between a first tube-voltage value and a second tube-voltage value lower than the first tube-voltage value;
updating the command value of the tube current at a predetermined timing according to switching of the tube voltage; and
specifying a filament-current command-value corresponding to the command value of the tube current based on the characteristic data.

15. The control method according to claim 14, further comprising applying a voltage to the filament based on the specified filament-current command-value, wherein:
the step of updating the command value of the tube current is updating the command value of the tube current, as the predetermined timing, when the tube voltage switches from the first tube-voltage value to the second tube-voltage value; and
the step of specifying a filament-current command-value is specifying a filament-current command-value corresponding to the command value of the tube current at a predetermined tube voltage based on the characteristic data.

16. The control method according to claim 14, wherein:
the step of updating the command value of the tube current is updating the command value of the tube current, as the predetermined timing, at a timing of switching of a view determined by the number of times of data acquisition per rotation of a rotating frame.
